(19) United States
(12) Patent Application Publication
(10) Pub. No.: US 2017/0112781 A1
(43) Pub. Date: Apr. 27, 2017

(54) TRANSDERMAL DRUG DELIVERY SYSTEMS WITH POLYISOBUTYLENE FACE ADHESIVE

(71) Applicant: Noven Pharmaceuticals, Inc., Miami, FL (US)

(72) Inventors: Takito Shima, Miami, FL (US); Jun LIAO, Miami, FL (US)

(73) Assignee: Noven Pharmaceuticals, Inc., Miami, FL (US)

(21) Appl. No.: 15/334,506

(22) Filed: Oct. 26, 2016

Related U.S. Application Data
(60) Provisional application No. 62/246,886, filed on Oct. 27, 2015.

(51) Int. Cl. A61K 9/70; A61K 31/27; A61K 47/32; A61K 9/00

(52) U.S. Cl. CPC A61K 9/7084; A61K 9/0014; A61K 31/27; A61K 47/32

(57) ABSTRACT

Described are transdermal drug delivery systems for extended delivery of a therapeutic agent in the form of a flexible finite system for topical application, comprising (i) a polymer matrix comprising a therapeutic agent and (ii) a face adhesive layer disposed on a skin-contacting side of the polymer matrix comprising a rubber-based polymer. Methods of making and using such systems also are described.

### CROSS-REFERENCE TO RELATED APPLICATIONS

This application claims the benefits under 35 U.S.C. §119(e) to U.S. provisional application 62/246,886, filed Oct. 27, 2015, the contents of which are incorporated herein by reference in their entirety.

### FIELD OF THE INVENTION

Described herein are transdermal drug delivery systems suitable for long-term application. The transdermal drug delivery systems are useful, for example, for transdermally delivering therapeutic agents over an extended period of time, such as over a period of time of 3 days, 7 days, or longer.

### BACKGROUND

Many factors influence the design and performance of transdermal drug delivery compositions. These include the individual drugs themselves, the physical and chemical characteristics of the compositions' components and their performance and behavior relative to other components, external and environmental conditions during manufacturing and storage, properties of the application site, the desired rate of drug delivery and therapeutic onset, the desired drug delivery profile, and the intended duration of delivery, among others.

A major design choice in the preparation of a transdermal drug delivery composition relates to the structure of the system, e.g., the selection and identity of the one or more layers of the system, and to the polymer components of the layer(s), e.g., the polymers used in the drug-containing carrier layer and/or any non-drug containing polymer layers. Typically, the polymers are pressure-sensitive adhesives, but different pressure-sensitive adhesive polymers have different properties that make them more or less advantageous for use in a given composition. Factors considered when selecting polymers for use in a transdermal drug delivery composition may include, for example, the solubility of the drug(s) to be formulated in the polymer, whether the polymer includes any reactive moieties that may react with any reactive moieties of the drug or other components of the composition, the physical compatibility of the polymer with other components of the composition, the desired physical properties of the composition (e.g., tackiness and wear properties), the desired pharmacokinetic properties of the composition (e.g., the rate and duration of drug delivery), etc.

Transdermal drug delivery systems designed for application over an extended period of time, such as up to 3 days, up to 7 days, or longer, present additional challenges, including the need to load a sufficient amount of therapeutic agent for drug delivery over the extended time period, and the need to achieve the desired pharmacokinetic profile over the extended time period. For example, rate controlling membranes or counter-ions have been used to prevent a drug delivery profile that includes an initial “burst” of drug flux on the first day of application followed by depleted drug flux on subsequent days. In this context a “counter ion” refers to a counter ion to a functional group on the therapeutic agent. For example, a carboxyl group is a counter ion for tertiary amine-containing drugs, such as rivastigmine. For example, US 2014/0276478 describes transdermal drug delivery systems for tertiary amine drugs that include a drug-containing polymer matrix, rate-controlling membrane and face adhesive, where one or more of the layers includes a counter ion (e.g., a carboxyl group-containing compound). Still, there remains a need for transdermal drug delivery systems designed for application over an extended time period.

### SUMMARY

This invention relates generally to transdermal drug delivery systems, and more particularly, to transdermal drug delivery systems comprising a face adhesive layer comprising a rubber-based polymer, such as a rubber-based pressure-sensitive adhesive.

In accordance with some embodiments, there are provided transdermal drug delivery systems, comprising a drug-containing polymer matrix and a face adhesive layer disposed on the skin-contacting side of the drug-containing polymer matrix, wherein the face adhesive layer comprises a rubber-based polymer, such as a rubber-based pressure-sensitive adhesive.

In accordance with some embodiments, there are provided transdermal drug delivery systems for extended delivery of a therapeutic agent in the form of a flexible finite system for topical application, comprising (i) a polymer matrix comprising a therapeutic agent and (ii) a face adhesive layer disposed on a skin-contacting side of the polymer matrix comprising a rubber-based pressure-sensitive adhesive. In some embodiments, the rubber-based pressure-sensitive adhesive of the face adhesive layer comprises a rubber-based polymer selected from polyisobutylene polymers, styrene-isoprene-styrene polymers, and combinations thereof. In some embodiments, the rubber-based pressure-sensitive adhesive comprises at least 90%, or at least 95%, by dry weight of the face adhesive layer. In some embodiments, the face adhesive layer further comprises one or more components selected from tackifiers, plasticizers, fillers, antioxidants and penetration enhancers. In some embodiments, the face adhesive layer is free of acrylic polymers and is free of silicone polymers. In some embodiments, the therapeutic agent comprises one or more functional groups and the face adhesive layer is free of counter ions to the one or more functional groups, and/or the transdermal drug delivery system is free of counter ions to the one or more functional groups.

In some embodiments, the transdermal drug delivery system is formulated to achieve sustained delivery of the therapeutic agent over a period of time of at least 3 days, at least 4 days, or at least 7 days. In some embodiments, the transdermal drug delivery system is formulated to achieve a flux drop of less than 30% over an application period of at least 3 days, at least 4 days, or at least 7 days.

In some embodiments, the transdermal drug delivery system further comprises a backing layer and/or a release liner.

In accordance with some embodiments, there are provided methods of transdermally delivering a therapeutic agent, comprising applying a transdermal drug delivery system as described herein to the skin or mucosa of a subject in need thereof. In some embodiments, the transdermal drug delivery system is applied for a duration of at least 3 days, at least 4 days, or at least 7 days.

In accordance with some embodiments, there are provided transdermal drug delivery systems as described herein for use in transdermally delivering a therapeutic agent to a subject in need thereof.

In accordance with some embodiments, there are provided uses of rubber-based polymers in the preparation of a medicament for transdermally delivering a therapeutic agent to a subject in need thereof, wherein the medicament is in the form of a transdermal drug delivery system as described herein, having a face adhesive layer that comprises a rubber-based polymer.

In accordance with some embodiments, there are provided methods of preparing a transdermal drug delivery system for extended delivery of a therapeutic agent in the form of a flexible finite system for topical application, comprising applying a face adhesive layer comprising a rubber-based polymer on a skin-contacting side of a polymer matrix comprising the therapeutic agent.

### BRIEF DESCRIPTION OF THE DRAWINGS

FIG. 1 illustrates a transdermal delivery system as described herein that comprises a backing, a drug-in-adhesive polymer matrix layer, a face adhesive layer, and a release liner (when present).

FIG. 2 illustrates the drug flux of rivastigmine across human skin over 7 days from transdermal drug delivery systems having difference face adhesive layers, as compared to drug flux from a commercial rivastigmine patch (EXELON® Patch).

FIG. 3 illustrates the drug flux of rivastigmine across human skin over 7 days from a transdermal drug delivery system having a face adhesive layer as described herein, as compared to drug flux from a commercial rivastigmine patch (EXELON® Patch).

FIG. 4 illustrates the in vivo (mini-pig) plasma concentration of rivastigmine over 7 days from a transdermal drug delivery system as described herein, as compared to drug flux from a commercial rivastigmine patch (EXELON® Patch) applied daily for 7 days.

### DETAILED DESCRIPTION

In accordance with some embodiments, the present invention provides transdermal drug delivery systems comprising a face adhesive layer comprising a rubber-based polymer. In specific embodiments, the systems are suitable for application over an extended period of time, such as up to 3 days, up to 7 days, or longer. The present inventors surprisingly discovered that using a face adhesive comprising a rubber-based polymer can permit the preparation of transdermal drug delivery systems that exhibit satisfactory skin adhesion and sustained drug delivery over an extended period of time, such as up to 3 days, up to 7 days, or longer. In specific embodiments, the systems achieve a pharmacokinetic profile such that the decline in flux is less than 30% over a 7 day application period.

### DEFINITIONS

Technical and scientific terms used herein have the meanings commonly understood by one of ordinary skill in the art to which the present invention pertains, unless otherwise defined. Reference is made herein to various methodologies known to those of ordinary skill in the art. Publications and other materials setting forth such known methodologies to which reference is made are incorporated herein by reference in their entireties as though set forth in full. Any suitable materials and/or methods known to those of ordinary skill in the art can be utilized in carrying out the present invention. However, specific materials and methods are described. Materials, reagents and the like to which reference is made in the following description and examples are obtainable from commercial sources, unless otherwise noted.

As used herein, the singular forms “a,” “an,” and “the” designate both the singular and the plural, unless expressly stated to designate the singular only.

The term “about” means that the number comprehended is not limited to the exact number set forth, and is intended to refer to numbers around the number while not departing from the scope of the invention. As used herein, “about” will be understood by persons of ordinary skill in the art and will vary to some extent on the context in which it is used. If there are uses of the term which are not clear to persons of ordinary skill in the art given the context in which it is used, “about” will mean up to plus or minus 10% of the particular term.

The phrases “% by weight, based on the dry weight of the polymer matrix” and “% dry weight” and “% dry” refer to the weight of the component(s) in the finished polymer matrix, e.g., after drying and evaporation of volatile processing solvents.

The phrase “substantially free” as used herein generally means that the described composition (e.g., transdermal drug delivery system, polymer matrix, etc.) comprises less than about 5%, less than about 3%, or less than about 1% by weight, based on the total weight of the composition at issue, of the excluded component. The phrase “free of” as used herein means that the described composition (e.g., polymer matrix, etc.) is formulated without adding the excluded component(s) as an intended component, although trace amounts may be present in other components or as a by-product or contaminant, such that the composition comprises at most only trace amounts of the excluded component(s).

As used herein “subject” denotes any animal in need of drug therapy, including humans. For example, a subject may be suffering from or at risk of developing a condition that can be treated or prevented with a drug that is effective upon transdermal delivery, or may be taking such a drug for health maintenance purposes.

As used herein, the phrases “therapeutically effective amount” and “therapeutic level” mean that drug dosage or plasma concentration in a subject, respectively, that provides the specific pharmacological response for which the drug is administered in a subject in need of such treatment. It is emphasized that a therapeutically effective amount or therapeutic level of a drug will not always be effective in treating the conditions/diseases described herein, even though such dosage is deemed to be a therapeutically effective amount by those of skill in the art. For convenience only, exemplary dosages, drug delivery amounts, therapeutically effective amounts and therapeutic levels are provided below with reference to adult human subjects. Those skilled in the art can adjust such amounts in accordance with standard practices as needed to treat a specific subject and/or condition/disease.

As used herein, “active surface area” means the surface area of the drug-containing layer of the transdermal drug delivery system.

As used herein, “coat weight” refers to the weight of the layer at issue per unit area of the active surface area of the transdermal drug delivery system.

As used herein, “flux” (also called “permeation rate”) is defined as the absorption of a drug through skin or mucosal tissue, and is described by Fick's first law of diffusion:

J=−D(dCm/dx)

where J is the flux in g/cm²/sec, D is the diffusion coefficient of the drug through the skin or mucosa in cm²/sec and dCₘ/dx is the concentration gradient of the drug across the skin or mucosa.

As used herein, the term “transdermal” refers to delivery, administration or application of a drug by means of direct contact with skin or mucosa. Such delivery, administration or application is also known as dermal, percutaneous, transmucosal and buccal. As used herein, “dermal” includes skin and mucosa, which includes oral, buccal, nasal, rectal and vaginal mucosa.

As used herein, “transdermal drug delivery system” refers to a system (e.g., a device) comprising a composition that releases drug upon application to the skin (or any other surface noted above). Typically, the transdermal drug delivery system is a substantially non-aqueous, solid form, capable of conforming to the surface with which it comes into contact, and capable of maintaining such contact so as to facilitate topical application without adverse physiological response, and without being appreciably decomposed by aqueous contact during topical application to a subject. Many such systems are known in the art and commercially available, such as transdermal drug delivery patches. In accordance with specific embodiments described herein, the transdermal drug delivery systems comprise a drug-containing polymer matrix that comprises a pressure-sensitive adhesive or bioadhesive and drug, and a face adhesive layer disposed on the skin-contacting side of the drug-containing polymer matrix that is adopted for application to a user's (e.g., a subject's) skin.

As used herein, “polymer matrix” refers to a polymer composition which contains one or more drugs. In some embodiments, the matrix comprises a pressure-sensitive adhesive polymer or a bioadhesive polymer. In other embodiments, the matrix does not comprise a pressure-sensitive adhesive or bioadhesive. As used herein, a polymer is an “adhesive” if it has the properties of an adhesive per se, or if it functions as an adhesive by the addition of tackifiers, plasticizers, crosslinking agents or other additives. Thus, in some embodiments, the polymer matrix comprises a pressure-sensitive adhesive polymer or a bioadhesive polymer, with drug dissolved or dispersed therein. The polymer matrix also may comprise tackifiers, plasticizers, crosslinking agents, enhancers, co-solvents, fillers, antioxidants, solubilizers, crystallization inhibitors, or other additives described herein. U.S. Pat. No. 6,024,976 describes polymer blends that are useful in accordance with the transdermal systems described herein. The entire contents of U.S. Pat. No. 6,024,976 is incorporated herein by reference.

As used herein, the term “pressure-sensitive adhesive” refers to a viscoelastic material which adheres instantaneously to most substrates with the application of very slight pressure and remains tacky. A polymer is a pressure-sensitive adhesive within the meaning of the term as used herein if it has the properties of a pressure-sensitive adhesive per se or functions as a pressure-sensitive adhesive by admixture with tackifiers, plasticizers or other additives.

The term pressure-sensitive adhesive also includes mixtures of different polymers and mixtures of polymers, such as polyisobutylenes (PIB), of different molecular weights, wherein each resultant mixture is a pressure-sensitive adhesive. In the last case, the polymers of lower molecular weight in the mixture are not considered to be “tackifiers,” said term being reserved for additives which differ other than in molecular weight from the polymers to which they are added.

In some embodiments, the polymer matrix is a pressure-sensitive adhesive at room temperature and has other desirable characteristics for adhesives used in the transdermal drug delivery art. Such characteristics include good adherence to skin, ability to be peeled or otherwise removed without substantial trauma to the skin, retention of tack with aging, etc. In some embodiments, the polymer matrix has a glass transition temperature (Tg), measured using a differential scanning calorimeter, of between about −70° C. and 0° C.

As used herein, the term “rubber-based pressure-sensitive adhesive” refers to a viscoelastic material which has the properties of a pressure-sensitive adhesive and which contains at least one natural or synthetic elastomeric polymer.

In some embodiments, the transdermal drug delivery system includes one or more additional layers, such as one or more additional polymer matrix layers, or one or more rate-controlling membranes, or one or more additional face adhesive layers that adhere the transdermal drug delivery system to the user's skin. In other embodiments, the transdermal drug delivery system includes a single drug-containing polymer matrix layer and a single face adhesive layer, and no rate-controlling membrane.

The transdermal drug delivery system also may include a drug impermeable backing layer or film. In some embodiments, the backing layer is adjacent one face of the polymer matrix layer. When present, the backing layer protects the polymer matrix layer (and any other layers present) from the environment and prevents loss of the drug and/or release of other components to the environment during use. Materials suitable for use as backing layers are well-known known in the art and can comprise films of polyester, polyethylene, vinyl acetate resins, ethylene/vinyl acetate copolymers, polyvinyl chloride, polyurethane, and the like, metal foils, non-woven fabric, cloth and commercially available laminates. A typical backing material has a thickness in the range of 2 to 1000 micrometers. For example, 3M Scotchpak™ 1012 or 9732 backing material (a polyester film with an ethylene vinyl acetate copolymer heat seal layer) may be used for the transdermal drug delivery systems described herein.

The transdermal drug delivery system also may include a release liner, typically located adjacent the opposite face of the system as compared to the backing layer. When present, the release liner is removed from the system prior to use to expose the polymer matrix layer and/or a face adhesive layer prior to topical application. Materials suitable for use as release liners are well-known known in the art and include commercially available products such as fluoropolymer-coated polyester films sold by 3M under the Scotchpak™ brand, such as Scotchpak™ 1020, 1022, 9744, 9748 and 9749, and polyester films with silicone release coatings such as SYL-OFF® Advantage Series Silicone Release Coatings sold by Dow Corning Corporation.

In specific embodiments, the transdermal drug delivery system consists of a backing, a single drug-containing polymer matrix layer, a single face adhesive layer, and, optionally, a release liner that is removed prior to use.

The transdermal drug delivery system may be packaged or provided in a package, typically a peelable pouch, as is used in the art for transdermal drug delivery systems in general. A wide variety of materials known to those skilled in the art of transdermal drug delivery may be used as pouchstock materials, including Surlyn® packaging resins (ethylene acid copolymers) sold by DuPont®, Wilmington, Del.

Face Adhesive

The transdermal drug delivery systems described herein may comprise a skin-contacting face adhesive layer, separate from the drug-containing polymer matrix. In some embodiments, the face adhesive comprises a rubber-based polymer, such as a rubber-based pressure sensitive adhesive, such as a polyisobutylene or styrene-isoprene-styrene polymer, optionally formulated with a tackifier or plasticizer to provide pressure-sensitive adhesive properties.

Examples of suitable rubber-based polymers and rubber-based pressure-sensitive adhesives include natural or synthetic polyisoprene, polybutylene, polyisobutylene, styrene-butadiene polymers, styrene-isoprene-styrene block copolymers, hydrocarbon polymers, such as butyl rubber, halogen-containing polymers, such as polyacrylic-nitrile, polytetrafluoroethylene, polyvinylchloride, polyvinylidene chloride, and polychlorodiene, and other copolymers thereof. Further specific examples include polyisobutylene pressure-sensitive adhesive polymers, such as DURO-TAK® 87-6908 (Henkel), which comprises polyisobutylene polymer and tackifier. Other specific examples include styrene-isoprene-styrene block copolymers, such as Kraton® D1111 KT (Kraton Performance Polymers, Inc.).

The face adhesive layer may comprise a single rubber-based pressure-sensitive adhesive, or a blend of two or more adhesives. The face adhesive layer may comprise one or more tackifiers, plasticizing agents (such as mineral oil, hydrogenated hydrocarbon resins, aliphatic resins, rosins, and terpenes), and/or other components selected to confer satisfactory physical stability, chemical stability, and/or skin adhesion, as discussed in more detail below.

The face adhesive layer may comprise at least 90%, at least 95%, at least 96%, at least 97%, at least 98%, or least 99% by dry weight of the one or more rubber-based pressure sensitive adhesives. In this context, “% by dry weight of the one or more rubber-based pressure sensitive adhesives” includes the dry weight of the rubber-based polymer and any tackifiers and/or plasticizers included in the composition.

In some embodiments, the face adhesive layer is free of counter ions to any functional groups on the therapeutic agent(s) being formulated in the system. In some embodiments, both the drug-containing polymer matrix layer and the face adhesive layer are free of counter ions to any functional groups on the therapeutic agent(s) being formulated in the system. In some embodiments the transdermal drug delivery system as a whole is free of counter ions to any functional groups on the therapeutic agent(s) being formulated in the system.

In some embodiments, the face adhesive layer is free of silicone polymers, including silicone pressure-sensitive adhesive polymers. In some embodiments, the face adhesive layer is free of acrylic polymers, including pressure-sensitive adhesive acrylic polymers. In some embodiments, the face adhesive layer is free of both silicone polymers and acrylic polymers, including being of free of both silicone pressure-sensitive adhesive polymers and pressure-sensitive adhesive acrylic polymers.

The face adhesive layer may be applied at a coat weight of from 2 to about 20 mg/cm², including at from about 5 to about 10 mg/cm², including 5 mg/cm², 6 mg/cm², 7 mg/cm², 8 mg/cm², 9 mg/cm², or 10 mg/cm², or any coat weight there between. As illustrated in the examples, the coat weight of the face adhesive layer can be selected and controlled to select and control drug delivery.

The polymer component(s) and/or coat weight of the face adhesive may be selected to achieve one or more of the following characteristics: good skin adhesion for the extended period of application (e.g., at least 3 days, at least 7 days, or longer); minimal resistance to drug diffusion; minimal solubility for the drug without exhibiting “dumping” upon initial contact with skin that leads to a “burst effect;” and/or physical and chemical compatibility with the drug.

As noted above, the inventors surprisingly found that using a face adhesive layer as described herein, comprising a rubber-based polymer, can permit the preparation of transdermal drug delivery systems that exhibit satisfactory skin adhesion and sustained drug delivery over an extended period of time, such as up to 3 days, up to 7 days, or longer. Thus, in some embodiments, the face adhesive functions not only to provide long-term skin adhesion, but also controls drug delivery to provide drug delivery over an extended period of time.

Drug-Containing Polymer Matrix

The compositions described herein comprise a polymer matrix that is suitable for use with the therapeutic agent (drug) being formulated. Typical polymers used in a polymer matrix for a transdermal drug delivery system include pressure-sensitive adhesive acrylic polymers, silicone-containing polymers, and rubber-based polymers (such as polyisobutylene and styrene-isoprene-styrene polymers). Non-limiting examples of suitable polymers are set forth below.

Acrylic Polymers

The term “acrylic polymer” is used here as in the art interchangeably with “polyacrylate,” “polyacrylic polymer,” and “acrylic adhesive.” Suitable acrylic-based polymers include homopolymers, copolymers, terpolymers, and the like of various acrylic acids or esters. In some embodiments, acrylic-based polymers are adhesive polymers. In other embodiments, acrylic-based polymers function as an adhesive by the addition of tackifiers, plasticizers, crosslinking agents or other additives. Often, acrylic polymers used in transdermal drug delivery systems include polymers of one or more monomers of acrylic acids and other copolymerizable monomers, copolymers of alkyl acrylates and/or methacrylates and/or copolymerizable secondary monomers or monomers with functional groups, and combinations of acrylic-based polymers based on their functional groups. Acrylic-based polymers having functional groups include copolymers and terpolymers which contain, in addition to nonfunctional monomer units, further monomer units having free functional groups, where the monomers can be monofunctional or polyfunctional. By varying the amount of each type of monomer added, the cohesive properties of the resulting acrylic polymer can be changed as is known in the art. In some embodiments, the acrylic polymer is composed of at least 50% by weight of an acrylate or alkyl acrylate monomer, from 0 to 20% of a functional monomer copolymerizable with the acrylate, and from 0 to 40% of other monomers.

Typical acrylate monomers include acrylic acid and methacrylic acid and alkyl acrylic or methacrylic esters such as methyl acrylate, ethyl acrylate, propyl acrylate, amyl acrylate, butyl acrylate, butyl methacrylate, hexyl acrylate, methyl methacrylate, hexyl methacrylate, heptyl acrylate, octyl acrylate, nonyl acrylate, 2-ethylbutyl acrylate, 2-ethylbutyl methacrylate, isooctyl acrylate, isooctyl methacrylate, 2-ethylhexyl acrylate, 2-ethylhexyl methacrylate, decyl acrylate, decyl methacrylate, dodecyl acrylate, dodecyl methacrylate, tridecyl acrylate, tridecyl methacrylate, glycidyl acrylate, and corresponding methacrylic esters.

Typical non-functional acrylic-based polymers include any acrylic based polymer having no or substantially no free functional groups.

As used herein, “functional monomers or groups,” are monomer units typically in acrylic-based polymers which have reactive chemical groups which modify the acrylic-based polymers directly or which provide sites for further reactions. Examples of functional groups include carboxyl, epoxy, hydroxyl, sulfoxyl, and amino groups. Acrylic-based polymers having functional groups contain, in addition to the nonfunctional monomer units described above, further monomer units having free functional groups. As noted above, the monomers can be monofunctional or polyfunctional. Typical carboxyl functional monomers include acrylic acid, methacrylic acid, itaconic acid, maleic acid, and crotonic acid. Typical hydroxyl functional monomers include 2-hydroxyethyl methacrylate, 2-hydroxyethyl acrylate, hydroxymethyl acrylate, hydroxymethyl methacrylate, hydroxyethyl acrylate, hydroxyethyl methacrylate, hydroxypropyl acrylate, hydroxypropyl methacrylate, hydroxybutyl acrylate, hydroxybutyl methacrylate, hydroxyamyl acrylate, hydroxyamyl methacrylate, hydroxyhexyl acrylate, hydroxyhexyl methacrylate. Others include amide-group containing-monomers such as octyl acrylamide, and vinyl group containing monomers, such as vinyl acetate and vinyl pyrrolidone In some embodiments, the functionality of the polymer(s) is selected depending on the drug being formulated, e.g., is selected based on whether the functional group is reactive or non-reactive with a functional group on the drug being formulated.

Further details and examples of acrylic adhesives which are suitable in the practice of the invention are described in Satas, “Acrylic Adhesives,” Handbook of Pressure-Sensitive Adhesive Technology, 2nd ed., pp. 396-456 (D. Satas, ed.), Van Nostrand Reinhold, New York (1989); “Acrylic and Methacrylic Ester Polymers,” Polymer Science and Engineering, Vol. 1, 2nd ed., pp 234-268, John Wiley & Sons, (1984); U.S. Pat. No. 4,390,520; and U.S. Pat. No. 4,994,267, all of which are expressly incorporated by reference in their entireties.

Suitable acrylic polymers also include pressure-sensitive adhesives which are commercially available, such as the acrylic-based adhesives sold under the trademarks DURO-TAK® and GELVA® by Henkel Corporation, Bridgewater, N.J. (such as DURO-TAK® 87-2516, -2287, -4287, -4098, -2852, -2196, -2296, -2194, -2516, -2070, -2353, -2154, -2510, -9085, -9088 and 73-9301; and GELVA® 2480, 788, 737, 263, 1430, 1753, 1151, 2450, 2495, 3067, 3071, 3087 and 3235). Other suitable acrylic adhesives include those sold under the trademark EUDRAGIT® by Evonik Industries AG, Essen Germany. For example, hydroxy functional adhesives with a reactive functional OH group can be used, including GELVA® 737, 788, and 1151, and DURO-TAK® 87-2287, -4287, -2510 and -2516.

Silicon Polymers

In some embodiments, the drug-containing polymer matrix comprises a silicone-based polymer. The term “silicone-based” polymer is used interchangeably with the terms silicon polymers, siloxane, polysiloxane, and silicones as used herein and as known in the art. A suitable silicone-based polymer may also be a pressure-sensitive adhesive. Thus, in some embodiments, the silicone-based polymer is an adhesive polymer. In other embodiments, the silicone-based polymer functions as an adhesive by the addition of tackifiers, plasticizers, crosslinking agents, or other additives.

Suitable polysiloxanes include silicone pressure-sensitive adhesives which are based on two major components: (i) a polymer or gum and (ii) a tackifying resin. A polysiloxane adhesive can be prepared by cross-linking a gum, typically a high molecular weight polydiorganosiloxane, with a resin, to produce a three-dimensional silicate structure, via a condensation reaction in an appropriate organic, volatile solvent, such as ethyl acetate or heptane. The ratio of resin to polymer can be adjusted in order to modify the physical properties of polysiloxane adhesives. Sobieski, et al., “Silicone Pressure Sensitive Adhesives,” Handbook of Pressure-Sensitive Adhesive Technology, 2nd ed., pp. 508-517 (D. Satas, ed.), Van Nostrand Reinhold, New York (1989).

Exemplary silicone-based polymers are adhesives (e.g., capable of sticking to the site of topical application), including pressure-sensitive adhesives. Illustrative examples of silicone-based polymers having reduced silanol concentrations include silicone-based adhesives (and capped polysiloxane adhesives) such as those described in U.S. Pat. No. Re. 35,474 and U.S. Pat. No. 6,337,086, which are incorporated herein by reference in their entireties, and which are commercially available from Dow Corning Corporation (Dow Corning Corporation, Medical Products, Midland, Mich.) as BIO-PSA® 7-4100, -4200 and -4300 product series, and non-sensitizing, pressure-sensitive adhesives produced with compatible organic volatile solvents (such as ethyl acetate or heptane) and available commercially under their BIO-PSA® 7-4400 series, -4500 series, such as -4502, and -4600 series.

Further details and examples of silicone pressure-sensitive adhesives which are useful in the polymer matrices and compositions and methods described herein are mentioned in the following U.S. Pat. Nos. 4,591,622; 4,584,355; 4,585,836; and 4,655,767, which are all expressly incorporated by reference herein in their entireties. It should also be understood that silicone fluids are also contemplated for use in the polymer matrices and methods described herein.

In some embodiments, the polymer matrix is free of silicone-containing acrylic polymers.

Rubber-Based Polymers

As noted above, in some embodiments the drug-containing polymer matrix comprises one or more rubber-based polymers, such as one or more rubber-based pressure-sensitive adhesives, such as any one or more of those discussed above in the context of the face adhesive.

Additionally or alternatively, the polymer matrix may comprise a non-adhesive polymer, such as ethyl cellulose.

Antioxidant

In accordance with any of the embodiments described herein, the polymer matrix and/or face adhesive layer may comprise an antioxidant. The antioxidant may be one known for use in transdermal drug delivery systems, such as butylhydroxytoluene (BHT), butylhydroxyanisole (BHA), tertiary-butylhydroquinone (TBHQ), ascorbic acid, ascorbyl palmitate, alpha-tocopherol and its esters, fumaric acid, malic acid, sodium ascorbate, sodium metabisulfite, and propyl gallate, and mixtures thereof. The antioxidant may comprise from about 0 to about 1%, including from about 0 to about 0.5% by weight of the polymer matrix face adhesive layer.

Other Components

In accordance with any of the embodiments described herein, the polymer matrix and/or face adhesive polymer may comprise one or more other pharmaceutically acceptable excipients, such as a plasticizer, penetration enhancer, filler, and the like. In some embodiments, the polymer matrix comprises from about 0% to about 20% of one or more such excipients.

A “penetration enhancer” is an agent known to accelerate the delivery of the drug through the skin. These agents also have been referred to as accelerants, adjuvants, and sorption promoters, and are collectively referred to herein as “enhancers.” This class of agents includes those with diverse mechanisms of action, including those which have the function of improving percutaneous absorption, for example, by changing the ability of the stratum corneum to retain moisture, softening the skin, improving the skin's permeability, acting as penetration assistants or hair-follicle openers or changing the state of the skin including the boundary layer.

Illustrative penetration enhancers include but are not limited to polyhydric alcohols such as dipropylene glycol, propylene glycol, and polyethylene glycol; oils such as olive oil, squalene, and lanolin; fatty ethers such as cetyl ether and oleyl ether; fatty acid esters such as isopropyl myristate; urea and urea derivatives such as allantoin which affect the ability of keratin to retain moisture; polar solvents such as dimethyidecylphosphoxide, methyloctylsulfoxide, dimethyllaurylamide, dodecylpyrrolidone, isosorbitol, dimethylacetonide, dimethylsulfoxide, decylmethylsulfoxide, and dimethylformamide which affect keratin permeability; salicylic acid which softens the keratin; amino acids which are penetration assistants; benzyl nicotinate which is a hair follicle opener; and higher molecular weight aliphatic surfactants such as lauryl sulfate salts which change the surface state of the skin and drugs administered. Other agents include oleic and linoleic acids, ascorbic acid, panthenol, butylated hydroxytoluene (BHT), tocopherol, tocopheryl acetate, tocopheryl linoleate, propyl oleate, and isopropyl palmitate.

In some embodiments, the polymer matrix or transdermal drug delivery system does not include a penetration enhancer. In some embodiments, the polymer matrix or transdermal drug delivery system does not include a penetration enhancer that includes carboxylic acid groups. Thus, in some embodiments, the polymer matrix or transdermal drug delivery system does not include a penetration enhancer that includes carboxylic acid groups.

In accordance with any of the embodiments described herein, the polymer matrix and/or face adhesive may further comprise one or more various thickeners, fillers, and other additives or components known for use in transdermal drug delivery systems to further modify properties of the matrix or face adhesive, such as polyvinylpyrrolidone (PVP), crosslinked PVP (crospovidone), polyvinylpyrrolidone/vinylacetate copolymers (PVP/VA, copovidone), ethylene-vinyl acetate copolymers, cellulose derivatives, silica, and other components.

In accordance with any of the embodiments described herein, the polymer matrix may comprise a humectant. Humectants suitable for use in transdermal drug delivery systems are known, and include PVP, crospovidone, copovidone, and combinations of any two or more thereof. The amount of humectant can be selected based on desired properties, such as an amount effective to impart desired physical properties, such as the adhesion properties of the polymer matrix, such as shear. In some embodiments, a humectant is used in an amount up from about 3% to about 10% dry weight of the polymer matrix, including about 3%, about 5% or about 10% dry weight.

Therapeutic Agents

The transdermal drug delivery systems described herein can be used for formulating any therapeutic agent, such as any therapeutic agent known or determined to be therapeutically active upon transdermal delivery. In some embodiments the therapeutic agent is one for which delivery over an extended period of time is desired, such as one for which delivery over 3 days, 7 days, or longer, is desired.

Examples of therapeutic agents include rivastigmine, selegiline, buprenorphine, rotigotine, oxybutynin, granisetron, scopolamine, clonidine, testosterone, estradiol, ethinyl estradiol, noregestromin, levonorgestrel, norethindrone acetate, norethindrone, gestodene, and fentanyl. These examples are provided as illustrative only, since the described transdermal drug delivery systems can be used for formulating any therapeutic agent.

In the embodiments described in the examples, the therapeutic agent is rivastigmine. Rivastigmine is a parasympathomimetic or cholinergic agent approved for the treatment of mild to moderate dementia of the Alzheimer's type and dementia due to Parkinson's disease. The drug can be administered orally or transdermally. The commercially available transdermal rivastigmine product (EXELON® Patch) is designed for daily use and comprises four layers: a backing layer, a polymer-drug matrix layer, and adhesive layer and a release liner. EXELON® Patch is available in three sizes, a 5 cm² patch that includes 9 mg rivastigmine and delivers about 4.6 mg rivastigmine in 24 hours, a 10 cm² patch that includes 18 mg rivastigmine and delivers about 9.5 mg rivastigmine in 24 hours, and a 15 cm² patch that includes 27 mg rivastigmine and delivers about 13.3 mg rivastigmine in 24 hours. (Doses of 9.5 mg/24 hours or 13.3 mg/24 hours are recommended effective doses.)

In some embodiments, the polymer matrix comprises from about 0.1% to about 50% by dry weight therapeutic agent, including from about 1% to about 30%, such as from about 5% to about 25% dry weight, of therapeutic agent.

Transdermal Drug Delivery Systems

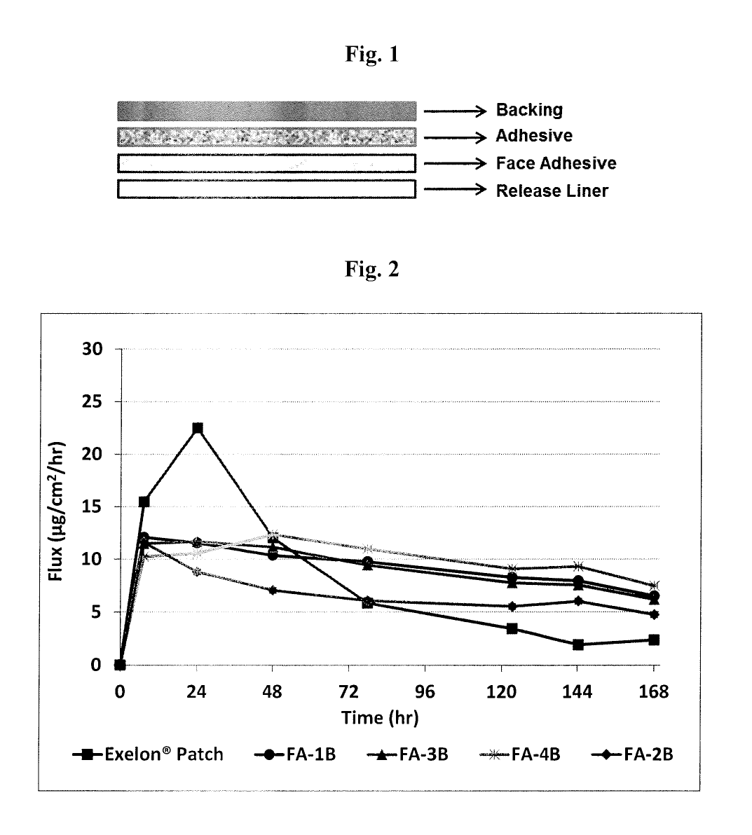

In some embodiments, the transdermal drug delivery system consists essentially of the polymer matrix layer and the face adhesive layer, as illustrated in FIG. 1. By “consists essentially of the polymer matrix layer and the face adhesive layer” means that the system does not contain any other layers that affect drug delivery, such as an additional face adhesive layer, rate-controlling polymer layer, rate-controlling membrane, or drug reservoir layer. It will be understood, however, that the system that consists essentially of the polymer matrix layer and face adhesive layer may comprise a backing layer and/or release liner. In other embodiments, the transdermal drug delivery system includes one or more other layers, such as one or more additional face adhesive layers, rate-controlling polymer layers, rate-controlling membranes, or drug reservoir layers.

The system may be of any shape or size suitable for transdermal application. The polymer matrices and face adhesive layers described herein may be prepared by methods known in the art, and formed into systems by methods known in the art. For example, the polymer matrix material can be applied to a backing layer and release liner by methods known in the art. For example, after the polymer matrix is formed, it may be brought into contact with a support layer, such a releaser liner layer or backing layer, in any manner known to those of skill in the art. Such techniques include calender coating, hot melt coating, solution coating, etc. The face adhesive layer also may be formed on a release liner, and then applied to the polymer matrix layer, and then the systems can be formed into sizes and shapes suitable for use

For example, a polymer matrix can be prepared by blending the components of the polymer matrix, applying the matrix material to a support layer such as a backing layer or release liner, and removing any remaining solvents, and a face adhesive layer can be prepared similarly. The order of steps, amount of ingredients, and the amount and time of agitation or mixing can be determined and optimized by the skilled practitioner. An exemplary general method is as follows:

Appropriate amounts of polymer(s), therapeutic agent(s), other component(s), and organic solvent(s) (for example toluene, or ethyl acetate and/or isopropyl alcohol) are combined and thoroughly mixed together in a vessel. The formulation is then transferred to a coating operation where it is coated onto a protective release liner at a controlled specified thickness. The coated product is then passed through an oven in order to drive off all volatile processing solvents. The dried product on the release liner is then joined to the backing material and wound into rolls for storage. A face adhesive solution containing the face adhesive in a suitable solvent may be coated onto a release liner and dried in a convection oven. The dried face adhesive on the release liner may be laminated with the prepared drug-in-adhesive polymer matrix (after removing its release liner) to form a laminate with a face adhesive. Appropriate size and shape “systems” are die-cut from the roll material and then pouched. Other manufacturing methods are known in the art that are suitable for making the systems described herein.

Also provided are methods of effecting transdermal drug delivery of a therapeutic agent, by applying a system as described herein to the skin or mucosa of a subject in need thereof. In some embodiments, the system is applied over a period of at least about 1 day, at least about 2 days, at least about 3 days, at least about 4 days, at least about 5 days, at least about 6 days, or at least about 7 days, such as for 1, 2, 3, 4, 5, 6 or 7 days. In some embodiments, the method is effective to achieve therapeutic levels of the therapeutic agent during the application period. In some embodiments, the method is effective to achieve a flux decline of less than 30% over the application period.

The following specific examples are included as illustrative of the transdermal drug delivery systems and polymer matrices described herein. These examples are in no way intended to limit the scope of the invention. Other aspects of the invention will be apparent to those skilled in the art to which the invention pertains.

### Example 1

Transdermal drug delivery systems were prepared using a polymer matrix comprised of 25% wt dry rivastigmine, 74% wt dry DURO-TAK® 87-2516 (pressure-sensitive adhesive acrylic polymer), and 1% wt dry BHT, applied at a coat weight of 10 mg/cm² to a polyester/ethylene vinyl acetate copolymer backing (Scotcpack 9732), and a face adhesive selected from the following:

| | | | FA-3B | FA-4B |
|---|---|---|---|---|
| | FA-1B | FA-2B | (▴) | (*) |
| | () | (♦) | (comparative) | (comparative) |
| PIB | 99.6% | 99.6% | 94.6% | 89.6% |
| (% wt dry) | | | | |
| BHT | 0.4% | 0.4% | 0.4% | 0.4% |
| (% wt dry) | | | | |
| EUDARGIT ® | — | — | 5.0% | 10.0% |
| L 100 | | | | |
| (% wt dry) | | | | |
| Coat Weight | 5 | 10 | 5 | 5 |
| (mg/cm²) | | | | |

PIB = DURO-TAK ® 87-6908
EUDRAGIT ® L 100 = anionic copolymer based on methacrylic acid and methyl methacrylate in a 1:1 ratio, having carboxyl groups which act as counter ions to the rivastigmine tertiary amine group.

Drug flux through human cadaver skin over 7 days was assessed, with results set forth in FIG. 2, as compared to EXELON® Patch (▪) having 18 mg rivastigmine per 10 cm². As seen in the figure, similar drug flux was observed from all systems with a face adhesive applied at a coat weight of 5 mg/cm². That is, the systems with a face adhesive comprising a polyisobutylene polymer and no counter ion achieved similar drug flux over the 7-day period as the systems with face adhesives comprising a polyisobutylene polymer and counter ion (EUDRAGIT® L 100). Drug flux from the system with a face adhesive applied at a coat weight of 10 mg/cm² was lower than drug flux from the comparable system with a face adhesive applied at a coat weight of 5 mg/cm². This indicates that the face adhesive may provide a rate controlling function, and that drug flux could be selected and controlled by selecting and controlling the coat weight of the face adhesive layer. The results also show that drug flux from the systems described herein was sustained and controlled over the 7-day application period.

### Example 2

Transdermal drug delivery systems were prepared using a polymer matrix comprised of 25% wt dry rivastigmine, 74% wt dry DURO-TAK® 87-2516 (pressure-sensitive adhesive acrylic polymer), and 1% wt dry BHT, applied at a coat weight of 15 mg/cm² to a polyester/ethylene vinyl acetate copolymer backing (Scotcpak™ 9732), and a face adhesive (FA-5B) comprised of 99.6% wt dry DURO-TAK® 87-6908 pressure-sensitive adhesive polyisobutylene polymer, 0.4% BHT, applied at a coat weight of 8 mg/cm².

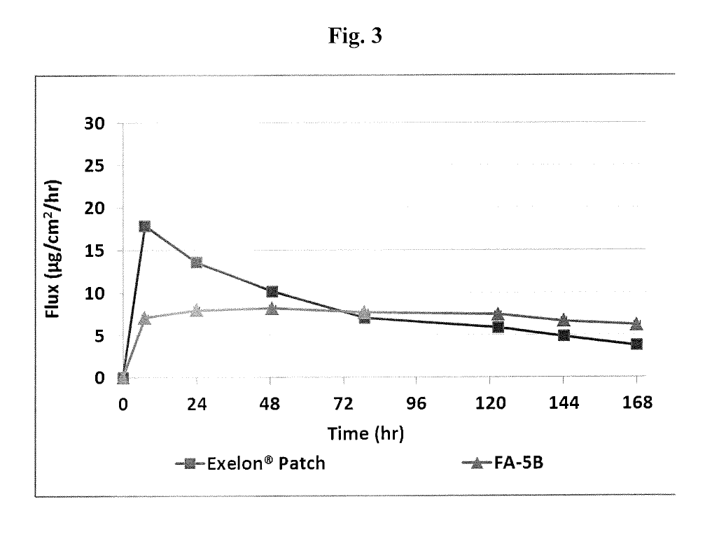

Drug flux through human cadaver skin over 7 days was assessed, with results set forth in FIG. 3 (▴), as compared to EXELON® Patch (▪) having 18 mg rivastigmine per 10 cm². As seen in the figure, drug flux from the system as described herein was sustained and controlled over the 7-day application period. The flux decline (Jₘₐₓ−J_{last (168 hrs)}) was less than 30%.

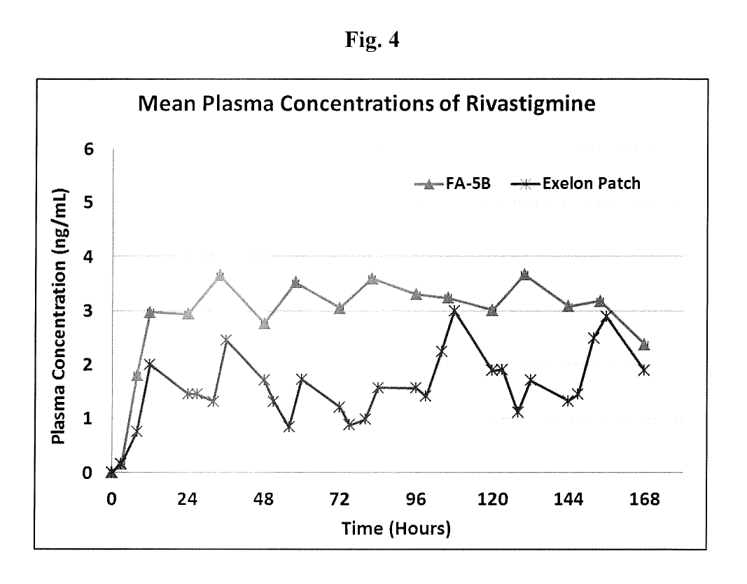

The transdermal drug delivery systems prepared as described above having 112.5 mg rivastigmine per 30 cm² were assessed in vivo in mini-pigs over a 7-day application period. Results (plasma concentration of rivastigmine) are set forth in FIG. 4 (▴), as compared to EXELON® Patch (*) having 18 mg rivastigmine per 10 cm² applied daily for 7 days. The results show that the rivastigmine plasma concentration achieved with the system described herein was sustained over the 7-day application period, and the steady-state plasma concentration was greater than that achieved with daily application of the EXELON® Patch.

What is claimed is:

1. A transdermal drug delivery system for extended delivery of a therapeutic agent in the form of a flexible finite system for topical application, comprising (i) a polymer matrix comprising a therapeutic agent and (ii) a face adhesive layer disposed on a skin-contacting side of the polymer matrix comprising a rubber-based polymer.

2. The transdermal drug delivery system of claim 1, wherein the rubber-based polymer of the face adhesive layer is selected from polyisobutylene polymers, styrene-isoprene-styrene polymers, and combinations thereof.

3. The transdermal drug delivery system of claim 1, wherein the face adhesive layer comprises a rubber-based pressure-sensitive adhesive comprising the rubber-based polymer and, optionally, one or more tackifiers and/or plasticizers.

4. The transdermal drug delivery system of claim 3, wherein the rubber-based pressure-sensitive adhesive comprises at least 90% by dry weight of the face adhesive layer.

5. The transdermal drug delivery system of claim 3, wherein the rubber-based pressure-sensitive adhesive comprises at least 95% by dry weight of the face adhesive layer.

6. The transdermal drug delivery system of claim 3, wherein the face adhesive layer further comprises one or more components selected from fillers, antioxidants and penetration enhancers.

7. The transdermal drug delivery system of claim 1, wherein the face adhesive layer is free of acrylic polymers and is free of silicone polymers.

8. The transdermal drug delivery system of claim 1, wherein the therapeutic agent comprises one or more functional groups and the face adhesive layer is free of counter ions to the one or more functional groups.

9. The transdermal drug delivery system of claim 1, wherein the therapeutic agent comprises one or more functional groups and the transdermal drug delivery system is free of counter ions to the one or more functional groups.

10. The transdermal drug delivery system of claim 1, formulated to achieve sustained delivery of the therapeutic agent over a period of time of at least 3 days, at least 4 days, or at least 7 days.

11. The transdermal drug delivery system of claim 1, formulated to achieve a flux drop of less than 30% over an application period of at least 3 days, at least 4 days, or at least 7 days.

12. The transdermal drug delivery system of claim 1, further comprising a backing layer.

13. The transdermal drug delivery system of claim 1, further comprising a release liner.

14. A method of transdermally delivering a therapeutic agent, comprising applying a transdermal drug delivery system according to claim 1 to the skin or mucosa of a subject in need thereof.

15. The method of claim 14, wherein the transdermal drug delivery system is applied for a duration of at least 3 days, at least 4 days, or at least 7 days.

16. A method of preparing a transdermal drug delivery system for extended delivery of a therapeutic agent in the form of a flexible finite system for topical application, comprising applying a face adhesive layer comprising a rubber-based polymer on a skin-contacting side of a polymer matrix comprising the therapeutic agent.
